(19) United States
(12) Patent Application Publication
(10) Pub. No.: US 2026/0357179 A1
(43) Pub. Date: Nov. 12, 2026

(54) DISPOSABLE FIXED BED FOR ADHERENT CELL CULTURE

(71) Applicant: TOFFLON LIFE SCIENCE CO., LTD, Shanghai (CN)

(72) Inventors: Minggang Sun, Shanghai (CN); Xiaohu You, Shanghai (CN); Yashuang Zhang, Shanghai (CN); Jinsheng Cheng, Shanghai (CN); Kun Xu, Shanghai (CN); Lei Zhu, Shanghai (CN); Xiangqi Kong, Shanghai (CN)

(21) Appl. No.: 19/125,755

(22) PCT Filed: Oct. 26, 2023
(86) PCT No.: PCT/CN2023/126806

§ 371 Date: Apr. 30, 2025

(30) Foreign Application Priority Data
Nov. 1, 2022 (CN) 202211355215.6

(51) Int. Cl. C12M 1/12; C12M 1/00; C12M 1/06; C12M 1/34

(52) U.S. Cl. CPC C12M 25/18; C12M 23/28; C12M 23/38; C12M 27/02; C12M 29/00; C12M 41/12

(57) ABSTRACT

Provided is a disposable fixed bed for adherent cell culture, comprising a pump casing, an upper cover plate, an annular pressing plate, a carrier, an annular bottom plate, a first supporting seat, a second supporting seat, a sealing connection mechanism, and a magnetic stirring mechanism. The annular bottom plate, the first supporting seat, and the second supporting seat are sequentially arranged on the pump casing from bottom to top. The upper cover plate is in a sealed connection to the pump casing. Stirring blades of the magnetic stirring mechanism are sleeved on a connecting column at the bottom of the pump casing. The first supporting seat and the second supporting seat jointly form one carrier supporting seat. The annular pressing plate and an annular groove structure of the carrier supporting seat jointly form one accommodating space. The carrier is arranged in the accommodating space.

### FIELD OF THE INVENTION

The invention relates to the field of cell culture technology, in particular to a disposable fixed bed for adherent cell culture.

### BACKGROUND OF THE INVENTION

Adherent-dependent cells need to attach to the wall of the culture vessel (such as bottle) during culture. Once attached, the cells quickly spread out, begin mitosis, and soon enter the logarithmic growth phase. Generally, after a few days, the cells will cover the entire culture surface and form a dense cell monolayer. Traditional adherent cell culture equipment has some drawbacks: large footprint; labor-intensive operation; mostly non-sealed equipment, which is prone to contamination; the shear force from stirring can damage cells; and low oxygen exchange efficiency. Therefore, there is an urgent need for a more efficient and suitable device for adherent cell culture.

### SUMMARY OF THE INVENTION

The purpose of the invention is to provide a disposable fixed bed for adherent cell culture to solve the problems of large footprint, labor-intensive operation, susceptibility to contamination, cell damage from stirring shear force, and low oxygen exchange efficiency in existing disposable adherent cell culture equipment.

To solve the above technical problems, the invention provides a disposable fixed bed for adherent cell culture, which includes: a pump housing, an upper cover plate, an annular pressure plate, a carrier, an annular base plate, a first support seat, a second support seat, a first sealing connection mechanism, and a magnetic stirring mechanism;
the annular base plate, the first support seat, and the second support seat are sequentially installed on the inner bottom surface of the pump housing from bottom to top, the upper cover plate is arranged at the open end of the pump housing and is sealingly connected to the pump housing through the first sealing connection mechanism, forming a first sealed space between the upper cover plate and the pump housing; the magnetic stirring mechanism includes a driving component and stirring blades that rotate under the drive of the driving component, the inner bottom surface of the pump housing has a connecting column, the stirring blades are sleeved on the connecting column, and the stirring blades and the connecting column are both located within the central through-hole of the annular base plate;
the first support seat and the second support seat each have a bottom wall, a side wall, and a top wall connected in sequence, a first preset gap is formed between the side wall of the first support seat and the side wall of the second support seat to form a first fluid channel, and the top wall of the second support seat is higher than the top wall of the first support seat;
the bottom of the first support seat abuts against the outer edge of the annular base plate, the bottom of the second support seat abuts against the bottom wall of the first support seat, the first support seat and the annular base plate enclose to form a first cavity, the first support seat and the second support seat enclose to form a second cavity, and the second support seat and the upper cover plate enclose to form a third cavity;
the first cavity is connected to the bottom of the pump housing through the central through-hole of the annular base plate, the top wall of the first support seat is provided with a fluid through-hole, the first cavity is connected to the second cavity through the fluid through-hole, the bottom wall or side wall of the second support seat is a grid structure, the second cavity is connected to the third cavity through the grid holes on the second support seat, the first support seat and the second support seat together form a carrier support seat, a second preset gap is formed between the outer side wall of the carrier support seat and the side wall of the pump housing to form a second fluid channel, and the third cavity is connected to the bottom of the pump housing through the second fluid channel;
the annular pressure plate is arranged on the carrier support seat and forms a containment space together with the annular groove structure of the carrier support seat, and the carrier is placed within the containment space.

Optionally, in the disposable fixed bed for adherent cell culture, the annular pressure plate and the bottom wall of the second support seat are both grid structures, the grid holes on the bottom wall of the second support seat are the fluid inlet of the containment space, and the grid holes on the annular pressure plate are the fluid outlet of the containment space.

Optionally, in the disposable fixed bed for adherent cell culture, the side wall of the second support seat is a grid structure, a third preset gap is formed between the annular pressure plate and the outer side wall of the carrier support seat, the grid holes on the side wall of the second support seat are the fluid inlet of the containment space, and the third preset gap between the annular pressure plate and the carrier support seat is the fluid outlet of the containment space.

Optionally, in the disposable fixed bed for adherent cell culture, the distance between the outer side wall of the carrier support seat and the carrier is greater than the third preset gap.

Optionally, in the disposable fixed bed for adherent cell culture, the carrier is formed by stacking multiple annular thin sheet structures.

Optionally, in the disposable fixed bed for adherent cell culture, the bottom wall of the first support seat gradually slopes downward from the abutment with the second support seat, and the distance between the bottom wall of the first support seat and the bottom wall of the second support seat gradually increases along the slope direction.

Optionally, in the disposable fixed bed for adherent cell culture, the edge of the top wall of the second support seat has a descending step, the edge of the annular pressure plate is arranged on the step surface of the descending step, and is fixedly connected to the step surface of the descending step through a connecting component.

Optionally, in the disposable fixed bed for adherent cell culture, the first support seat has a first sidewall, a bottom wall, a second sidewall, and a top wall connected sequentially, and the annular groove structure of the carrier support seat is formed by the first sidewall of the first support seat and the bottom wall and sidewall of the second support seat; alternatively, the second support seat has a first sidewall, a bottom wall, a second sidewall, and a top wall connected sequentially, and the annular groove structure of the carrier support seat is formed by the first sidewall, bottom wall, and second sidewall of the second support seat.

Optionally, in the disposable fixed bed for adherent cell culture, both the first support seat and the second support seat are integrally formed from a non-toxic rigid material.

Optionally, in the disposable fixed bed for adherent cell culture, both the pump housing and the upper cover plate are integrally formed from a non-toxic and transparent plastic material, and the material of the first support seat and the second support seat is the same as that of the pump housing and the upper cover plate.

Optionally, in the disposable fixed bed for adherent cell culture, the bottom center of the pump housing is an upward protrusion, and the interior of the upward protrusion has an accommodation space for arranging a driving component of the magnetic stirring mechanism. The inner bottom surface of the pump housing is provided with at least two positioning columns, and the annular base plate is correspondingly provided with at least two engaging holes, with the tops of the positioning columns inserted into the engagement holes of the annular base plate. In the first support seat, a chamfer structure is provided at the junction of the bottom wall and the sidewall, and an inclined pressing surface is disposed on the outer edge of the annular base plate, which is compatible with the chamfer structure.

Optionally, in the disposable fixed bed for adherent cell culture, it further includes a support column with threaded structures on both ends. The connecting column of the pump housing is provided with a first installation hole, and one end of the support column is inserted into the first installation hole and fixedly connected to the pump housing through threading. The top wall of the first support seat is provided with a second installation hole, and the other end of the support column is inserted into the second installation hole and fixedly connected to the first support seat through threading.

Optionally, in the disposable fixed bed for adherent cell culture, the open end of the pump housing extends radially outward to form a first annular flange, and the open end of the upper cover plate extends radially outward to form a second annular flange, the first annular flange and the second annular flange are arranged opposite to each other and are both provided with bolt holes;
the first sealing connection mechanism comprises: a first flexible sealing ring, two flexible compression rings and a first bolt-nut assembly; the first flexible sealing ring is disposed between the first annular flange and the second annular flange, the two flexible compression rings are respectively disposed on the end surface of the first annular flange facing away from the second annular flange and the end surface of the second annular flange facing away from the first annular flange, and the pump housing, the two flexible compression rings and the upper cover plate are fixedly connected by the first bolt and nut assembly.

Optionally, in the disposable fixed bed for adherent cell culture, it further includes a heat-preserving base and a second sealing connection mechanism. The pump housing is disposed on the heat-preserving base and is sealingly connected to the upper end edge of the heat-preserving base through the second sealing connection mechanism. A gap is left between the heat-preserving base and the pump housing to form a second enclosed space, which serves as a water bath temperature control chamber.

Optionally, in the disposable fixed bed for adherent cell culture, the top of the heat-preserving base is provided with an outwardly turned flange with multiple connecting through-holes, and the sidewall of the pump housing is provided with a bent portion corresponding to the outwardly turned flange, with multiple screw sleeves provided on the bent portion. The bent portion of the pump housing rests against the outwardly turned flange of the heat-preserving base, and the inner surface of the bent portion is a slanted surface. The second sealing connection mechanism includes a second flexible sealing ring, a flexible gasket, and a second bolt and nut assembly. The second flexible sealing ring is disposed between the bent portion of the pump housing and the outwardly turned flange of the heat-preserving base, the flexible gasket is disposed on the side of the outwardly turned flange facing away from the bent portion, and the heat-preserving base, the flexible gasket, and the pump housing are fixedly connected through the second bolt and nut assembly.

Optionally, in the disposable fixed bed for adherent cell culture, the bottom surface of the heat-preserving base is provided with multiple recessed structures of equal height, which are symmetrically distributed around the central axis of the connecting column.

Optionally, in the disposable fixed bed for adherent cell culture, it further includes a liquid inlet, a liquid outlet, a first electrode seat, a second electrode seat, and a third electrode seat, which are respectively disposed on the upper cover plate.

Optionally, in the disposable fixed bed for adherent cell culture, both the top wall of the second support seat and the annular compression ring are provided with electrode through-holes. The bottoms of the first electrode seat and the third electrode seat are respectively inserted into the corresponding electrode through-holes of the annular compression ring and are flush with the lower end surface of the annular compression ring. The bottom of the second electrode seat passes through a limiting ring and is inserted into the electrode through-hole of the second support seat, with the upper end surface of the limiting ring resting against a stepped surface of the second electrode seat and the lower end surface of the limiting ring resting against the top wall of the second support seat.

### ADVANTAGES OF THE INVENTION

The culture medium is operated in a perfusion mode, eliminating the need for centrifugal force during the harvest period. From cell thawing to the harvest of the final product, this equipment requires only one step, significantly simplifying the early stages of the culture process.

A magnetic stirring mechanism is built into the bottom, with the stirring blade of the magnetic stirring mechanism located outside the cell growth space and not in contact with the cultured cells, greatly reducing the shear force experienced by the cells.

A specific structure (second fluid channel) allows for curtain-style oxygen exchange, not only improving the volumetric oxygen dissolution coefficient but also enhancing the mixing uniformity of materials and temperature control stability.

The entire fixed bed (including the pump housing, upper cover plate, carrier, carrier support seat, etc.) is used as a disposable consumable, reducing the risk of contamination and increasing the culture area. A footprint of 0.64 square meters can provide a culture area of approximately 500 square meters, equivalent to 3,000 cell culture roller bottles (each with 1,700 square centimeters), not only improving work efficiency but also reducing the inoculation density and thus the cost of culturing the target cells.

### BRIEF DESCRIPTION OF THE DRAWINGS

FIG. 1 is the quarter-sectional view of the disposable fixed-bed device for adherent cell culture in Embodiment 1.

FIG. 2 is the exploded view of the disposable fixed-bed device for adherent cell culture in Embodiment 1.

FIG. 3 is the half-sectional view of the disposable fixed-bed device for adherent cell culture in Embodiment 1.

FIG. 4 is the half-sectional view of another embodiment of the disposable fixed-bed device for adherent cell culture.

FIG. 5 is the half-sectional view of yet another embodiment of the disposable fixed-bed device for adherent cell culture.

FIG. 6 is the stereoscopic view of the embodiment of the disposable fixed-bed device for adherent cell culture.

FIG. 7 is the quarter-sectional view of the disposable fixed-bed device for adherent cell culture in Embodiment 2.

FIG. 8 is the half-sectional view of the disposable fixed-bed device for adherent cell culture in Embodiment 2.

In the figures: 1. heat-preserving base; 2. flexible washer; 3. pump housing; 4. flexible compression ring; 5. upper cover plate; 6. first electrode seat; 7. second electrode seat; 8. third electrode seat; 9. annular compression plate; 10. carrier; 11. support column; 12. stirring blade; 13. annular base plate; 14. first support seat; 15. second support seat; 16. sealing ring; 17. liquid inlet; 18. liquid outlet; 19. limiting ring; 1a. recessed structure; 3a. positioning column; 3b. connecting column.

### DETAILED DESCRIPTION OF THE EMBODIMENTS

The technical solution of the present invention will be described clearly and completely below with reference to the accompanying drawings. In the description of the present invention, it should be noted that the terms “center,” “upper,” “lower,” “left,” “right,” “vertical,” “horizontal,” “inner,” “outer,” etc., indicate orientations or positional relationships based on the orientations or positional relationships shown in the drawings, and are only for the convenience of describing the present invention and simplifying the description, rather than indicating or implying that the referred device or element must have a specific orientation, be constructed and operated in a specific orientation, and thus should not be construed as limiting the present invention. In addition, the terms “first,” “second,” “third,” etc., are used for descriptive purposes only and should not be construed as indicating or implying relative importance.

The present invention will be further described below with reference to the accompanying drawings.

### Embodiment 1

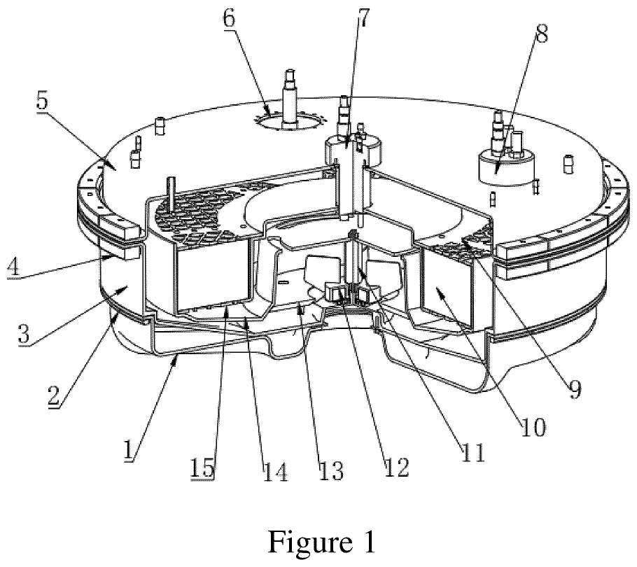

Please refer to FIGS. 1 to 3, which are schematic structural figures of the disposable fixed bed for adherent cell culture according to an embodiment of the present invention. As shown in FIGS. 1-3, the disposable fixed bed for adherent cell culture includes: a pump housing 3, an upper cover plate 5, an annular compression plate 9, a carrier 10, an annular base plate 13, a first support seat 14, a second support seat 15, a sealing connection mechanism, and a magnetic stirring mechanism. The annular base plate 13, the first support seat 14, and the second support seat 15 are sequentially installed from bottom to top on the inner bottom surface of the pump housing 3. The upper cover plate 5 is arranged at the open end of the pump housing 3 and is sealingly connected to the pump housing 3 through the sealing connection mechanism, forming a first sealed space between the upper cover plate 5 and the pump housing 3. The magnetic stirring mechanism includes a driving component (not labeled in the figures) and a stirring blade 12 that rotates under the drive of the driving component. The inner bottom surface of the pump housing 3 has a connecting column 3b, and the stirring blade 12 is sleeved on the connecting column 3b. Both the stirring blade 12 and the connecting column 3b are located within the central through-hole of the annular base plate 13. The first support seat 14 and the second support seat 15 each has a first side wall, a bottom wall, a second side wall, and a top wall (not labeled in the figures) connected in sequence. The second support seat 15 has a bottom wall, a side wall, and a top wall connected in sequence. A first preset gap is formed between the second side wall of the first support seat 14 and the side wall of the second support seat 15 to form a first fluid channel. The top wall of the second support seat 15 is higher than the top wall of the first support seat 14. The bottom of the first support seat 14 abuts against the outer edge of the annular base plate 13, and the bottom of the second support seat 15 abuts against the bottom wall of the first support seat 14. The first support seat 14 and the annular base plate 13 enclose to form a first cavity, the first support seat 14 and the second support seat 15 enclose to form a second cavity, and the second support seat 15 and the upper cover plate 5 enclose to form a third cavity. The first cavity is in fluid communication to the bottom of the pump housing 3 through the central through-hole of the annular base plate 13. A fluid through-hole is opened on the top wall of the first support seat 14, and the first cavity is in fluid communication to the second cavity through the fluid through-hole. The bottom wall of the second support seat 15 is of a grid structure, and the second cavity is in fluid communication the third cavity through the grid structure on the bottom wall of the second support seat 15. A second preset gap is formed between the first side wall of the first support seat 14 and the side wall of the pump housing 3 to form a second fluid channel. The third cavity is in fluid communication to the bottom of the pump housing 3 through the second fluid channel. The annular compression plate 9 is of a grid structure and is fixedly arranged on the second support seat 15. Together with the first side wall of the first support seat 14, the bottom wall and side wall of the second support seat 15, they form a containment space. The carrier 10 is arranged within the containment space.

Specifically, the annular base plate 13 is provided with a central through-hole (not labeled in the figures) and at least two engagement holes (not labeled in the figure). The diameter of the central through-hole is required to be larger than the diameter of the stirring blade 12. The inner bottom surface of the pump housing 3 is provided with at least two positioning columns 3a, which correspond one-to-one with the engagement holes on the annular base plate 13. The top ends of the positioning columns 3a are inserted into the engagement holes of the annular base plate 13, thereby achieving a snap-fit connection between the annular base plate 13 and the bottom of the pump housing 3.

Preferably, the at least two engagement holes are uniformly distributed along the circumference of the annular base plate 13, and the positioning columns 3a are integrally formed with the pump housing 3.

The first support seat 14 and the second support seat 15 together form a carrier support seat. The outer side wall (i.e., the first side wall of the first support seat 14) of the carrier support seat and the side wall of the pump housing 3 form a second fluid channel. The carrier support seat has an upwardly open annular groove structure, which, together with the annular compression plate 9, forms a containment space. The carrier 10 is uniformly loaded within the containment space.

In this embodiment, the carrier 10 is composed of multiple structural units arranged in a disordered manner. The structural units may be strip-shaped, block-shaped, sheet-shaped, or other shapes, which are not limited here. The multiple structural units should be loaded as uniformly as possible within the containment space, and the overall structure of the carrier 10 should match the containment space.

Preferably, the carrier 10 is formed by stacking annular thin-sheet structures. The layered structure design of the carrier 10 can avoid the problem of uneven distribution of the carrier within the containment space.

In this embodiment, the second support seat 15 has only one side wall, so it needs to cooperate with the first side wall of the first support seat 14 to form an upwardly open annular groove structure. That is, the annular groove structure of the carrier support seat is formed by the first side wall of the first support seat 14 and the bottom wall and side wall of the second support seat 15. Accordingly, the containment space for the carrier 10 is defined by the annular compression plate 9, the first support seat 14, and the second support seat 15. The outer side wall of the carrier support seat is the first side wall of the first support seat 14.

In other embodiments, the second support seat 15 may adopt a structure similar to that of the first support seat 14, having two side walls. That is, the second support seat 15 has a first side wall, a bottom wall, a second side wall, and a top wall connected in sequence. The annular groove structure of the carrier support seat is formed by the first side wall, bottom wall, and second side wall of the second support seat 15. Accordingly, the containment space for the carrier 10 is defined by the annular compression plate 9 and the first side wall, bottom wall, and second side wall of the second support seat 15. Since the second support seat 15 already has an annular groove structure that cooperates with the annular compression plate 9, the first support seat 14 may have two side walls or only one side wall. While the first support seat 14 has two side walls, the outer side wall of the carrier support seat is still the first side wall of the first support seat 14, and the first side wall of the second support seat 15 and the first side wall of the first support seat 14 may be tightly fitted by an interference fit to improve the firmness of the assembly of the two support seats. While the first support seat 14 has only one side wall, the outer side wall of the carrier support seat is the first side wall of the second support seat 15, and the outer side wall of the carrier support seat (i.e., the first side wall of the second support seat 15) and the side wall of the pump housing 3 form a second fluid channel. To prevent the second support seat 15 from moving left and right, a limiting structure needs to be provided at the edge of the bottom wall of the first support seat 14.

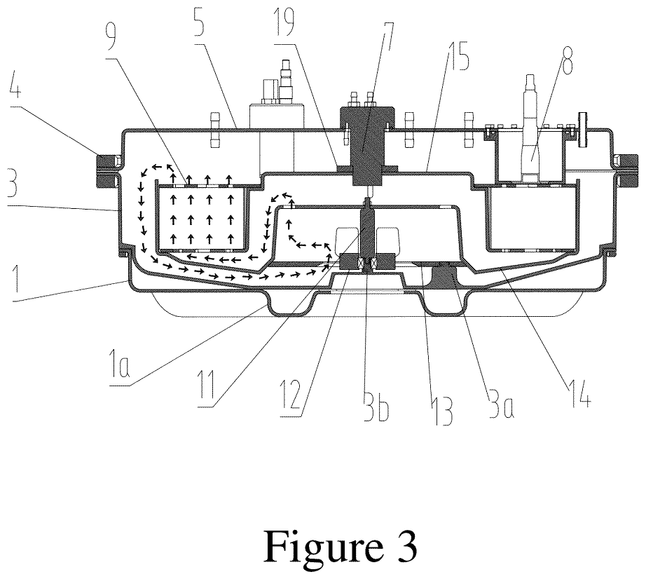

Please continue to refer to FIG. 3. In the first support seat 14, a chamfer structure (not labeled in the figures) is provided at the junction of the bottom wall and the second side wall. The outer edge of the annular base plate 13 is correspondingly provided with an inclined pressing surface (not labeled in the figures), which matches the chamfer structure. The bottom of the first support seat 14 abuts against the inclined pressing surface of the annular base plate 13, and the bottom of the second support seat 15 abuts against the bottom wall of the first support seat 14. Under the action of gravity and stress, the first support seat 14 and the second support seat 15 are fixed in position and not easily separated. The two may be fixedly connected by an interference fit, making them even more difficult to separate.

Wherein, the bottom wall of the first support seat 14 gradually slopes downward from the abutment with the second support seat 15, and the end of the bottom wall of the first support seat 14 that is not in contact with the second support seat 15 (i.e., the harvesting point) maintains a certain distance from the bottom wall of the second support seat 15. The bottom wall of the second support seat 15 may be horizontally arranged or inclined, which is not limited here.

Preferably, the bottom wall of the first support seat 14 is inclined, and the distance between the bottom wall of the first support seat 14 and the bottom wall of the second support seat 15 gradually increases along the inclined direction. In this way, the harvesting point is located at the lowest part of the carrier support seat, ensuring complete harvesting without residue when performing cell harvesting work from the harvesting point.

Preferably, the inner edge of the annular base plate 13 is set as an inclined surface, which forms an angle with the upper surface of the annular base plate 13 to facilitate the smooth flow of fluid from the bottom of the pump housing 3 into the first cavity enclosed by the annular base plate 13 and the first support seat 14.

In this embodiment, the annular compression plate 9 is of a grid structure with multiple grid holes, which penetrate the annular compression plate 9 in the thickness direction of the annular compression plate 9. The edge of the top wall of the second support seat 15 has a descending step, and the edge of the annular compression plate 9 is placed on the step surface of the descending step. Both the step surface of the descending step and the edge of the annular compression plate 9 are provided with through-holes for inserting connecting components. The edge of the annular compression plate 9 is fixedly connected to the step surface of the second support seat 15 through connecting components (such as screw and nut assemblies). The outer side wall of the carrier support seat (i.e., the first side wall of the first support seat 14) is required to be slightly higher than the height of the annular compression plate 9.

As shown in FIG. 3, the annular compression plate 9 is fixedly arranged on the second support seat 15 and, together with the first side wall of the first support seat 14, the bottom wall and side wall of the second support seat 15, form a containment space, which is the cell growth space. The fluid (e.g., culture medium) in the containment space can smoothly flow out through the multiple grid holes on the annular compression plate 9. The carrier 10 is arranged within the containment space and is made of medical polymer material or fiber material, which is of a porous structure.

Please continue to refer to FIG. 1. The pump housing 3 and the upper cover plate 5 are generally disk-shaped structures. The height of the pump housing 3 is greater than that of the upper cover plate 5. The upper cover plate 5 is covered on the pump housing 3. The open end of the pump housing 3 extends radially outward to form a first annular flange, and the open end of the upper cover plate 5 extends radially outward to form a second annular flange. The first annular flange and the second annular flange are arranged opposite to each other and are both provided with bolt holes. The first sealing connection mechanism includes a first flexible sealing ring (not labeled in the figures), two flexible compression rings 4, and a first bolt and nut assembly (not labeled in the figures). The first flexible sealing ring is arranged as a sealing component between the first annular flange and the second annular flange, in contact with the inner end surfaces of the first annular flange and the second annular flange. The two flexible compression rings 4 are respectively arranged on the outer end surfaces of the first annular flange and the second annular flange, i.e., one flexible compression ring 4 is arranged on the first annular flange of the pump housing 3 and located on the side away from the upper cover plate 5, and the other flexible compression ring 4 is arranged on the second annular flange of the upper cover plate 5 and located on the side away from the pump housing 3. Both flexible compression rings 4 are provided with multiple connecting holes. The bolt of the first bolt and nut assembly passes through the bolt holes on the pump housing 3 and the upper cover plate 5 and the connecting holes on the two flexible compression rings 4 to connect with the nut. After tightening, the bolt and nut assembly applies a mutually approaching pressure to the pump housing 3 and the upper cover plate 5, and the first flexible sealing ring is compressed and fixed between the pump housing 3 and the upper cover plate 5, thereby forming the first sealed space between the upper cover plate 5 and the pump housing 3. During the fixing process, the two flexible compression rings 4 can protect the edge structures of the pump housing 3 and the upper cover plate 5.

In this embodiment, the pump housing 3 and the upper cover plate 5 are sealingly connected through the bolt and nut assembly and the flexible sealing ring. In other embodiments, the connection between the pump housing 3 and the upper cover plate 5 may adopt other structural forms, and other sealing connection manners may be used between them, as long as the pump housing 3 and the upper cover plate 5 can be sealingly connected to form the first sealed space. For example, the edge of the upper cover plate 5 may be of a ring-shaped flanging structure, and the ring-shaped flanging structure and the ring-shaped flange structure of the pump housing 3 may be fixed by a snap-fit manner. The sealing component may be arranged in the ring-shaped groove of the ring-shaped flanging structure or the ring-shaped flange structure.

As shown in FIGS. 1-3, the disposable fixed bed for adherent cell culture further includes a support column 11. Both ends of the support column 11 are provided with threaded structures. The bottom of the pump housing 3 is provided with a connecting column 3b, which is vertically arranged. The top of the connecting column 3b is provided with a first installation hole (not labeled in the figures). One end of the support column 11 is inserted into the first installation hole and fixedly connected to the pump housing 3 through a threaded connection. The top wall of the first support seat 14 is provided with a second installation hole (not labeled in the figures). The other end of the support column 11 is inserted into the second installation hole and fixedly connected to the top wall of the first support seat 14 through a threaded connection manner.

In this embodiment, the support column 11 is connected between the first support seat 14 and the pump housing 3, which fixes the first support seat 14 to the pump housing 3 to prevent the first support seat 14 from moving up and down. And the lower step surface of the support column 11 abuts against the stirring blade 12, limiting the height of the stirring blade 12 on the connecting column 3b, so that the stirring blade 12 cannot move up and down.

It should be noted that the arrangement and fixing manner of the support column 11 described above are only examples and not limitations. In other embodiments, the support column 11 may be fixed between the first support seat 14 and the pump housing 3 by other manners, instead of providing threaded structures to both ends of the support column 11. The disposable fixed bed for adherent cell culture may not be provided with a support column 11, or the upper end of the support column 11 may not be fixed.

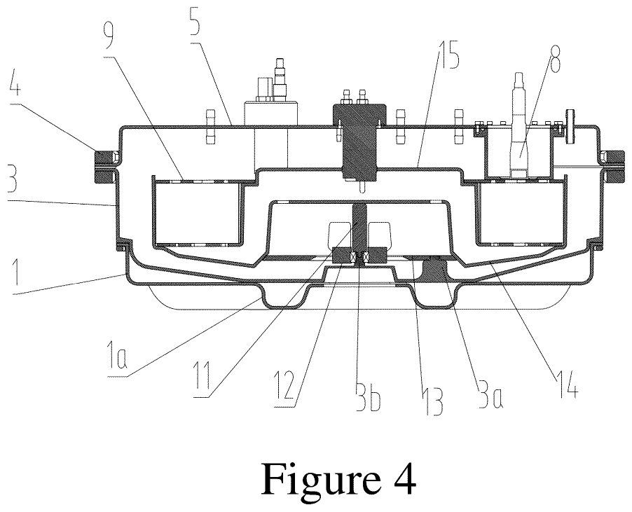

As shown in FIG. 4, in another embodiment, the lower end of the support column 11 is fixedly connected to the pump housing 3 through a threaded connection manner, and the upper end of the support column 11 is not fixedly connected to the first support seat 14 but abuts against the top wall of the first support seat 14.

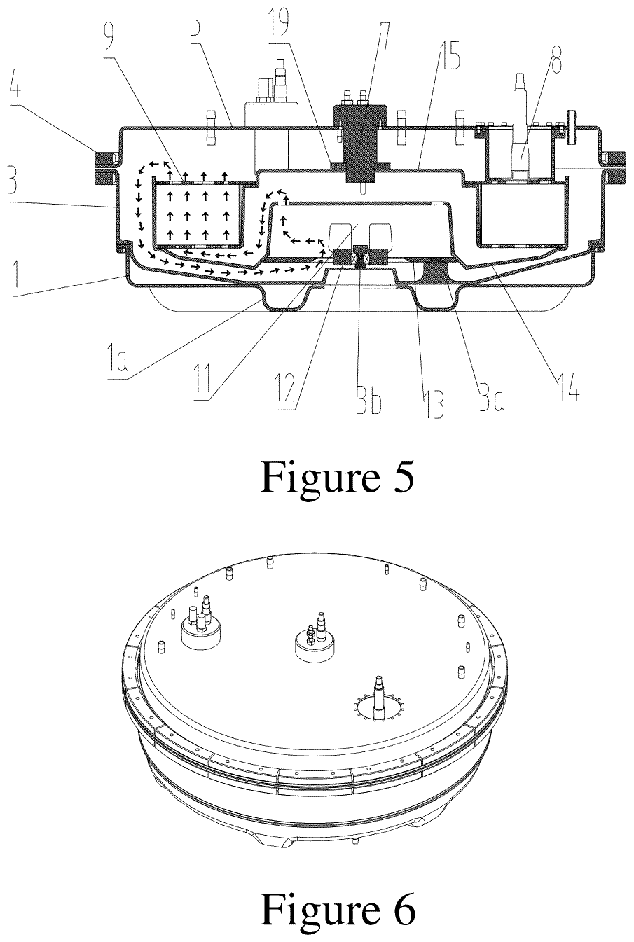

As shown in FIG. 5, in yet another embodiment, the stirring blade 12 is directly fixed by a compression nut (not labeled in the figures), so that it cannot move up and down on the connecting column 3b. While the rigidity of material of the first support seat 14 and the pump housing 3 is sufficient, the support column 11 may not be provided.

Please continue to refer to FIG. 1. The disposable fixed bed for adherent cell culture includes a heat-preserving base 1 and a second sealing connection mechanism. The upper edge of the heat-preserving base 1 is provided with an outwardly flanging edge, which is provided with multiple connecting through-holes. The side wall of the pump housing 3 is provided with a bent portion corresponding to the flanging edge, and the bent portion is provided with multiple screw sleeves. The multiple screw sleeves correspond one-to-one with the multiple connecting through-holes. The pump housing 3 is installed on the heat-preserving base 1, and its bent portion presses against the flanging edge of the heat-preserving base 1. The second sealing connection mechanism includes a second flexible sealing ring (not labeled in the figures), a flexible washer 2, and a second bolt-nut assembly (not labeled in the figures). The second flexible sealing ring is placed between the bent portion of the pump housing 3 and the flanging edge of the heat-preserving base 1 as a sealing element. The flexible washer 2 is placed on the side of the flanging edge opposite to the bent portion to protect the connection structure when the heat-preserving base 1 and the pump housing 3 are fixed together. The heat-preserving base 1, the flexible washer 2, and the pump housing 3 are fixed together by the second bolt-nut assembly.

Wherein, a gap is left between the heat-preserving base 1 and the pump housing 3 to form a second sealed space, which serves as a water bath temperature control chamber for introducing pure water. By controlling the water temperature in the second sealed space, the temperature of the culture medium in the first sealed space can be controlled.

In this embodiment, the flanging edge of the heat-preserving base 1 is horizontally arranged, and accordingly, the outer side of the bent portion of the pump housing 3 (i.e., the side in contact with the flanging edge) is a horizontal surface. The pump housing 3 and the heat-preserving base 1 are fixed together by a bolt-nut assembly and sealed by a flexible sealing ring.

In other embodiments, the connection between the pump housing 3 and the heat-preserving base 1 may adopt other structural forms, and other sealing connection manners may be used, as long as the pump housing 3 and the heat-preserving base 1 can be sealed to form the second sealed space. For example, the flanging edge of the heat-preserving base 1 and the outer side of the bent portion of the pump housing 3 may both be inclined, and the pump housing 3 and the heat-preserving base 1 may be sealed and fixed by sealant, welding, or other manners.

Preferably, the inner side of the bent portion of the pump housing 3 is inclined to ensure that no liquid accumulates in the bent portion.

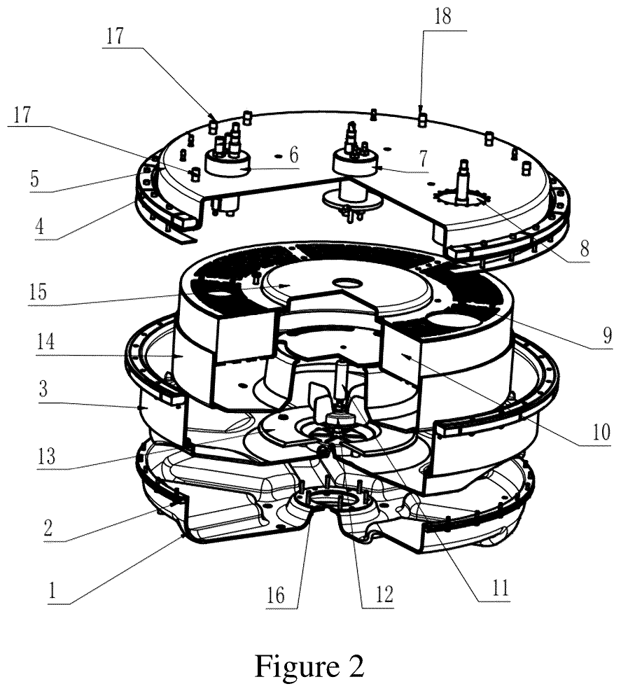

Please continue to refer to FIGS. 1 and 2, both the pump housing 3 and the heat-preserving base 1 are provided with an upward protrusion (not labeled in the figures) at the center. The upward protrusion of the heat-preserving base 1 corresponds to the upward protrusion of the pump housing 3, and a sealing ring 16 is placed between them. The upward protrusions of the heat-preserving base 1 and the pump housing 3 are both hollow to facilitate the installation of the driving components of the magnetic stirring mechanism. The connecting column 3b is located on the upward protrusion of the pump housing 3, and the stirring blade 12 of the magnetic stirring mechanism is sleeved on the connecting column 3b. A certain distance is maintained between the upward protrusion of the pump housing 3 and the annular base plate 13 to allow fluid (e.g., culture medium) to pass through smoothly.

Please continue to refer to FIG. 3, the bottom surface of the heat-preserving base 1 is provided with several recessed structures 1a. The heights of the several recessed structures 1a are equal and symmetrically distributed around the central axis of the connecting column 3b. The several recessed structures 1a are used to position the heat-preserving base 1 and the pump housing 3 to facilitate the alignment and installation of the stirring blade 12.

Please continue to refer to FIGS. 1 and 3, the disposable fixed bed for adherent cell culture also includes a first electrode seat 6, a second electrode seat 7, a third electrode seat 8, a liquid inlet 17, and a liquid outlet 18. The liquid inlet 17 and liquid outlet 18 are both located on the upper cover plate 5. The upper surface of the upper cover plate 5 is provided with threaded holes at the center and on two sides of the center. The outer walls of the first electrode seat 6, the second electrode seat 7, and the third electrode seat 8 are threaded. The first electrode seat 6, the second electrode seat 7, and the third electrode seat 8 are inserted into the corresponding threaded holes and fixed to the upper cover plate 5 by threaded connection.

In this embodiment, the first electrode seat 6 is used to detect pH and D0 values, the second electrode seat 7 is used to detect temperature and D0 values, and the third electrode seat 8 is used to detect biomass (i.e., cell density).

In this embodiment, both the annular compression plate 9 and the top wall of the second support seat 15 are provided with electrode through-holes. The bottoms of the first electrode seat 6 and the third electrode seat 8 are inserted into the electrode through-holes of the annular compression plate 9, and the bottom of the second electrode seat 7 is inserted into the electrode through-hole of the second support seat 15.

Preferably, the bottoms of the first electrode seat 6 and the third electrode seat 8 are flush with the lower surface of the annular compression plate 9.

Preferably, the bottom of the second electrode seat 7 passes through a limiting ring 19 and is inserted into the electrode through-hole of the second support seat 15. The lower end of the second electrode seat 7 has a stepped structure. The upper surface of the limiting ring 19 abuts against the stepped surface of the second electrode seat 7, and the lower surface of the limiting ring 19 abuts against the top wall of the second support seat 15. As shown in FIGS. 3 and 5, the limiting ring 19 is clamped between the stepped surface of the second electrode seat 7 and the top wall of the second support seat 15, preventing the second support seat 15 from moving upward.

In this embodiment, the pump housing 3 and the upper cover plate 5 are sealed together to form a first sealed space. The driving components of the magnetic stirring mechanism are located outside the first sealed space, while the stirring blade 12 of the magnetic stirring mechanism is located inside the first sealed space. Although the carrier 10 is also located inside the first sealed space, it is in a different chamber from the stirring blade 12. As shown in FIGS. 3-5, the carrier 10 is located in the cell growth space, while the stirring blade 12 is located outside the cell growth space.

In this embodiment, the heat-preserving base 1, pump housing 3, upper cover plate 5, annular compression plate 9, support column 11, annular base plate 13, first support seat 14, and second support seat 15 are all made of rigid materials. Among them, the components in contact with biological fluids (i.e., culture medium) must be made of safe and non-toxic materials suitable for cell culture.

Preferably, the first support seat 14 and the second support seat 15 are integrally formed from non-toxic rigid materials. Furthermore, the first support seat 14, the second support seat 15, the pump housing 3, and the upper cover plate 5 are all integrally formed from non-toxic and transparent plastic materials (e.g., PETG).

Wherein PETG is a non-crystalline copolyester with high transparency, excellent processing and molding properties, and is safe and non-toxic.

Preferably, the heat-preserving base 1, pump housing 3, upper cover plate 5, annular compression plate 9, carrier 10, annular base plate 13, first support seat 14, and second support seat 15 are all centrally symmetrical structures. The upward protrusions of the heat-preserving base 1 and the pump housing 3, as well as the central axis of the connecting column 3b, are all located on the central axis of the symmetrical structure.

During use, the culture medium is introduced into the pump housing 3 through the liquid inlet 17 on the upper cover plate 5, and the magnetic stirring mechanism is activated. The stirring blade 12 rotates under the drive of the driving components, causing the culture medium to flow. The culture medium flows through the central hole of the annular base plate 13 from the bottom of the pump housing 3 into the first chamber formed by the annular base plate 13 and the first support seat 14. As the stirring blade 12 continues to operate, the culture medium fills the first chamber and then flows through the fluid through-hole on the top wall of the first support seat 14 into the second chamber formed by the first support seat 14 and the second support seat 15. The culture medium in the second chamber flows through the first fluid channel between the first support seat 14 and the second support seat 15 into the space between the bottom walls of the first support seat 14 and the second support seat 15, and then enters the accommodation space where the carrier 10 is located through the grid holes on the bottom wall of the second support seat 15. When the accommodation space is filled with culture medium, the culture medium flows out through the grid holes on the annular compression plate 9 and returns to the bottom of the pump housing 3 through the second fluid channel between the pump housing 3 and the first support seat 14. This cycle achieves perfusion culture.

The disposable fixed bed for adherent cell culture provided in this embodiment uses perfusion culture instead of batch culture. During the culture process, waste liquid can be discharged to prevent the accumulation of harmful metabolites. This not only helps achieve high cell culture density but also reduces subsequent processing steps. From thawing the cells to harvesting the final product, only one step is required, and finished cell recovery can be performed without operations such as centrifugation, greatly simplifying the operation process and facilitating industrialization.

Moreover, the disposable fixed bed for adherent cell culture adopts a sealed structure, enabling a fully enclosed integrated process from cell activation, infection, expansion to final product recovery, reducing the risk of bacterial contamination during the culture process. More importantly, since the stirring blade 12 and the carrier 10 are not in the same chamber, the shear force on the cells is very small when the stirring blade 12 stirs the culture medium to maintain a uniform flow, effectively protecting the cells and improving cell yield.

Additionally, during the perfusion culture process, the culture medium needs to flow through the first fluid channel between the second side wall of the first support seat 14 and the side wall of the second support seat 15, as well as the second fluid channel between the first side wall of the first support seat 14 and the side wall of the pump housing 3. When the culture medium flows through these two fluid channels, it not only achieves curtain-type oxygen exchange, effectively increasing the contact area between the gas and the culture medium, improving the volumetric oxygen transfer coefficient, and enabling high-density culture, but also enhances the temperature uniformity and mixing uniformity of the culture medium, making the culture environment more stable.

In this embodiment, the disposable fixed bed for adherent cell culture is flat in shape, occupying only 0.64 m², while the surface area of the carrier 10 can reach 500 m². That is, an area of 0.64 m² can provide a culture area of approximately 500 m², equivalent to 3000 cell culture roller bottles (each roller bottle has 1700 cm²).

Compared to existing cell culture equipment, the disposable fixed bed for adherent cell culture provided in this embodiment (including the pump housing, upper cover plate, carrier, carrier support seat, etc.) can be used as a whole as a disposable consumable, not only reducing the risk of contamination and increasing the culture area but also lowering the inoculation density and improving culture efficiency, thereby effectively reducing the cost of target cell culture.

### Embodiment 2

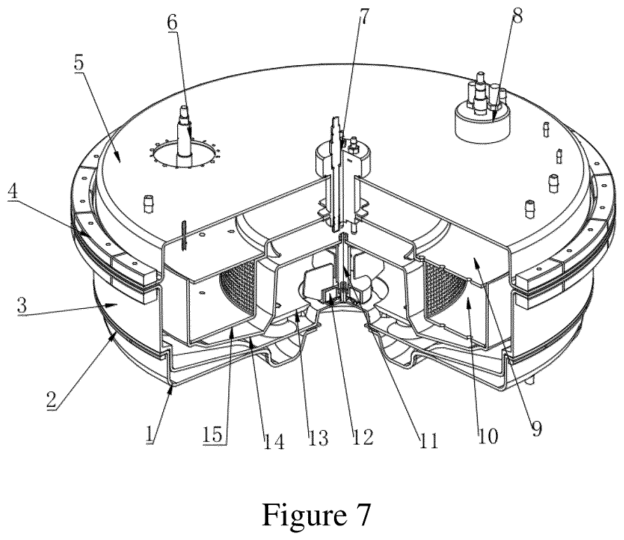

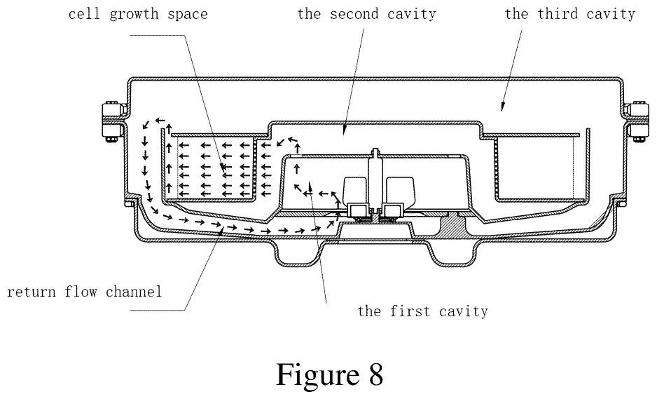

Referring to FIGS. 6 to 8, which are schematic figures of the disposable fixed bed for adherent cell culture according to the second embodiment of the present invention. As shown in FIGS. 6-8, the disposable fixed bed for adherent cell culture includes: a pump housing 3, an upper cover plate 5, an annular compression plate 9, a carrier 10, an annular base plate 13, a first support seat 14, a second support seat 15, a sealing connection mechanism, and a magnetic stirring mechanism. The annular base plate 13, the first support seat 14, and the second support seat 15 are installed sequentially from bottom to top on the inner bottom surface of the pump housing 3. The upper cover plate 5 is installed at the open end of the pump housing 3 and sealed with the pump housing 3 through the sealing connection mechanism, forming a first sealed space between the upper cover plate 5 and the pump housing 3. The magnetic stirring mechanism includes driving components (not labeled in the figures) and a stirring blade 12 that rotates under the drive of the driving components. The inner bottom surface of the pump housing 3 has a connecting column 3b, and the stirring blade 12 is sleeved on the connecting column 3b. Both the stirring blade 12 and the connecting column 3b are located in the central hole of the annular base plate 13. The first support seat 14 has a first side wall, a bottom wall, a second side wall, and a top wall (not labeled in the figures) connected in sequence. The second support seat 15 has a bottom wall, a side wall, and a top wall connected in sequence. A first preset gap is formed between the second side wall of the first support seat 14 and the side wall of the second support seat 15 to form a first fluid channel. The top wall of the second support seat 15 is higher than the top wall of the first support seat 14. The bottom of the first support seat 14 abuts against the outer edge of the annular base plate 13, and the bottom of the second support seat 15 abuts against the bottom wall of the first support seat 14. The first support seat 14 and the annular base plate 13 form a first chamber, the first support seat 14 and the second support seat 15 form a second chamber, and the second support seat 15 and the upper cover plate 5 form a third chamber. The first chamber is connected to the bottom of the pump housing 3 through the central hole of the annular base plate 13. The top wall of the first support seat 14 is provided with a fluid through-hole, and the first chamber is connected to the second chamber through the fluid through-hole. The side wall of the second support seat 15 has a grid structure, and the second chamber is connected to the third chamber through the grid holes on the side wall of the second support seat 15. A second preset gap is formed between the first side wall of the first support seat 14 and the side wall of the pump housing 3 to form a second fluid channel. The third chamber is connected to the bottom of the pump housing 3 through the second fluid channel. The annular compression plate 9 is fixed on the second support seat 15 and, together with the first side wall of the first support seat 14, the bottom wall and side wall of the second support seat 15, forms a containment space. The carrier 10 is placed in the containment space.

Specifically, the first support seat 14 and the second support seat 15 together form a carrier support seat. The annular compression plate 9 is fixed on the carrier support seat and, together with the annular groove structure of the carrier support seat, forms the containment space for the carrier 10.

Wherein, the side wall of the second support seat 15 has a grid structure, and a third preset gap is formed between the annular compression plate 9 and the outer side wall of the carrier support seat (i.e., the first side wall of the first support seat 14). The grid holes on the side wall of the second support seat 15 are the fluid inlet of the containment space, and the third preset gap between the annular compression plate 9 and the carrier support seat is the fluid outlet of the containment space. The biological fluid in the second chamber flows into the containment space through the side wall of the second support seat 15 and then flows out through the third preset gap.

The difference between this embodiment and Embodiment 1 lies in the flow direction of the culture medium in the containment space (i.e., the cell growth space). By matching with a suitable carrier 10, the culture quality can be further improved.

In Embodiment 1, both the bottom wall of the annular compression plate 9 and the second support seat 15 have grid structures. The grid holes on the bottom wall of the second support seat 15 are the fluid inlet of the containment space, and the grid holes on the annular compression plate 9 are the fluid outlet of the containment space. The fluid outlet and fluid inlet are located at the upper and lower ends of the cell growth space, respectively. When the stirring blade 12 drives the culture medium to flow, the culture medium in the cell growth space flows from bottom to top.

In this embodiment, the grid structure of the second support seat 15 is located on the side wall rather than the bottom wall. The annular compression plate 9 and the outer side wall of the carrier support seat (i.e., the first side wall of the first support seat 14) are not connected but have a certain gap to form a return flow channel. The annular compression plate 9 does not need to have a grid structure. When the stirring blade 12 drives the culture medium to flow, the culture medium in the cell growth space first flows horizontally, passes through the carrier 10, and then flows from bottom to top.

In this embodiment, the carrier 10 is composed of multiple annular thin sheet structures stacked together. This not only avoids the problem of uneven carrier distribution but also ensures that the annular thin sheet structures are basically horizontally arranged and the gaps between the annular thin plates are adapted to the flow direction of the culture medium, the culture medium in the cell growth space can flow through the gaps between the annular thin sheet structures, allowing cells to better absorb nutrients.

Preferably, the height of the carrier 10 is about 100 mm, and the thickness of the annular thin-plate structure is about 0.4 mm. Preferably, the spacing between the outer side wall of the carrier support seat (i.e., the first side wall of the first support seat 14) and the carrier 10 is greater than the third preset gap.

It should be noted that in this specification, each embodiment is described in a progressive manner. Each embodiment focuses on the differences from the other embodiments. The same or similar parts in the various embodiments can be referred to each other.

The above embodiments are merely used to illustrate the technical solutions of the present invention, rather than to limit it. Although the invention has been described in detail with reference to the aforementioned embodiments, ordinary technical people in the field should understand that they can still modify the technical solutions recorded in the aforementioned embodiments or make equivalent substitutions for some or all of the technical features therein. These modifications or substitutions do not deviate the essence of the corresponding technical solutions from the scope of the technical solutions of the embodiments of the present invention.

1. A disposable fixed bed for adherent cell culture, characterized in that it comprises: a pump housing (3), an upper cover plate (5), an annular compression plate (9), a carrier (10), an annular base plate (13), a first support seat (14), a second support seat (15), a first sealing connection mechanism, and a magnetic stirring mechanism;
the annular base plate (13), the first support seat (14), and the second support seat (15) are installed in sequence from bottom to top on the inner bottom surface of the pump housing (3), the upper cover plate (5) is disposed at the open end of the pump housing (3) and is sealingly connected to the pump housing (3) through the first sealing connection mechanism, forming a first sealed space between the upper cover plate (5) and the pump housing (3);
both the first support seat (14) and the second support seat (15) have a bottom wall, a side wall, and a top wall connected in sequence, with a first preset gap between the side wall of the first support seat (14) and the side wall of the second support seat (15) to form a first fluid channel, and the top wall of the second support seat (15) is higher than the top wall of the first support seat (14);
the bottom of the first support seat (14) abuts against the outer edge of the annular base plate (13), the bottom of the second support seat (15) abuts against the bottom wall of the first support seat (14), the first support seat (14) and the annular base plate (13) enclose to form a first cavity, the first support seat (14) and the second support seat (15) enclose to form a second cavity, and the second support seat (15) and the upper cover plate (5) enclose to form a third cavity;
the magnetic stirring mechanism comprises a driving component and a stirring blade (12) that rotates under the drive of the driving component; the first cavity accommodates the stirring blade (12);
the first cavity is in fluid communication to the bottom of the pump housing (3) through a central through-hole of the annular base plate (13), the top wall of the first support seat (14) is provided with a fluid through-hole, the first cavity is in fluid communication to the second cavity through the fluid through-hole, the side wall of the second support seat (15) is with grid holes, the second cavity is in fluid communication to the third cavity through the grid holes, the first support seat (14) and the second support seat (15) together constitute a carrier support seat, and a second preset gap is provided between the outer side wall of the carrier support seat and the side wall of the pump housing (3) to form a second fluid channel, the third cavity is in fluid communication to the bottom of the pump housing (3) through the second fluid channel;
the annular compression plate (9) is disposed on the carrier support seat and together with a ring-shaped groove structure of the carrier support seat form an accommodation space, and the carrier (10) is disposed within the accommodation space;
a third preset gap is provided between the annular compression plate (9) and the outer side wall of the carrier support seat, the grid structure on the side wall of the second support seat (15) serve as a fluid inlet of the accommodation space, and the third preset gap between the annular compression plate (9) and the carrier support seat serves as a fluid outlet of the accommodation space to guide culture medium to flow from the grid structure along the radial direction of the carrier (10) towards the fluid outlet and out of the accommodation space upwards from the fluid outlet.

2. The disposable fixed bed for adherent cell culture according to claim 1, characterized in that the top wall of the first support seat (14) provides the fluid through-hole to guide the culture medium upwards into the second cavity, the second cavity is used to adjust the flow state of the culture medium, the culture medium is in a turbulent state in the first cavity due to the action of the blade (12), and the culture medium flows in a laminar state in the accommodation space.

3. The disposable fixed bed for adherent cell culture according to claim 1, characterized in that a gap between the annular base plate (13) and the magnetic stirring mechanism serves as a culture medium inlet of the first cavity disposed near the stirring blade (12), and the fluid through-hole serves as a culture medium outlet of the first cavity disposed away from the stirring blade (12); the position of the stirring blade (12) is higher than the culture medium inlet, and the stirring blade (12) partially overlaps with the height of the carrier (10).

4. The disposable fixed bed for adherent cell culture according to claim 3, characterized in that the spacing between the outer side wall of the carrier support seat and the carrier (10) is greater than the third preset gap.

5. The disposable fixed bed for adherent cell culture according to claim 3, characterized in that the carrier (10) is formed by stacking multiple annular thin-plate structures, and gaps between the annular thin-plate structures are used to guide the culture medium to flow along the radial direction of the carrier (10).

6. The disposable fixed bed for adherent cell culture according to claim 1, characterized in that the bottom wall of the first support seat (14) gradually inclines downwards from the abutment with the second support seat (15), and the distance between the bottom wall of the first support seat (14) and the bottom wall of the second support seat (15) gradually increases along the direction of inclination.

7. The disposable fixed bed for adherent cell culture according to claim 1, characterized in that a descending step is provided at the edge of the top wall of the second support seat (15), the edge of the annular compression plate (9) is disposed on the step surface of the descending step and is fixedly connected to the step surface of the descending step via connecting components.

8. The disposable fixed bed for adherent cell culture according to claim 1, characterized in that the first support seat (14) has a first side wall, a bottom wall, a second side wall, and a top wall connected in sequence, and the ring-shaped groove structure of the carrier support seat is formed by the first side wall of the first support seat (14) and the bottom wall and side wall of the second support seat (15); or the second support seat (15) has a first side wall, a bottom wall, a second side wall, and a top wall connected in sequence, and the ring-shaped groove structure of the carrier support seat is formed by the first side wall, bottom wall, and second side wall of the second support seat (15).

9. The disposable fixed bed for adherent cell culture according to claim 1, characterized in that both the first support seat (14) and the second support seat (15) are integrally formed from a non-toxic rigid material.

10. The disposable fixed bed for adherent cell culture according to claim 9, characterized in that both the pump housing (3) and the upper cover plate (5) are integrally formed from a non-toxic and transparent plastic material, and the material of the first support seat (14) and the second support seat (15) is the same as that of the pump housing (3) and the upper cover plate (5).

11. The disposable fixed bed for adherent cell culture according to claim 1, characterized in that the center of the bottom surface of the pump housing (3) is of an upward protrusion, the interior of the upward protrusion has an accommodation space for arranging a driving component of the magnetic stirring mechanism, a connecting column (3b) is disposed on the top of the upward protrusion, and the stirring blade (12) is sleeved on the connecting column (3b);
at least two positioning columns (3a) are disposed on the inner bottom surface of the pump housing (3), at least two engagement holes are correspondingly disposed on the annular base plate (13), and the tops of the positioning columns (3a) are inserted into the engagement holes of the annular base plate (13);
in the first support seat (14), a chamfer structure is disposed at the junction of the bottom wall and the side wall, and an inclined pressing surface is disposed on the outer edge of the annular base plate (13), which is compatible with the chamfer structure.

12. The disposable fixed bed for adherent cell culture according to claim 1, characterized in that it further comprises a support column (11), both opposite ends of which are provided with threaded structures; a first mounting hole is opened on the connecting column (3b) of the pump housing (3), one end of the support column (11) is inserted into the first installation hole and fixedly connected to the pump housing (3) in a threaded connection manner, a second installation hole is opened on the top wall of the first support seat (14), and the other end of the support column (11) is inserted into the second installation hole and fixedly connected to the first support seat (14) in a threaded connection manner.

13. The disposable fixed bed for adherent cell culture according to claim 1, characterized in that the open end of the pump housing (3) extends radially outward to form a first annular flange, and the open end of the upper cover plate (5) extends radially outward to form a second annular flange, the first annular flange and the second annular flange are arranged opposite to each other and are both provided with bolt holes;
the first sealing connection mechanism comprises: a first flexible sealing ring, two flexible compression rings (4) and a first bolt-nut assembly; the first flexible sealing ring is disposed between the first annular flange and the second annular flange, the two flexible compression rings (4) are respectively disposed on the end surface of the first annular flange facing away from the second annular flange and the end surface of the second annular flange facing away from the first annular flange, and the pump housing (3), the two flexible compression rings (4) and the upper cover plate (5) are fixedly connected by the first bolt and nut assembly.

14. The disposable fixed bed for adherent cell culture according to claim 1, characterized in that it further comprises: a heat-preserving base (1) and a second sealing connection mechanism; the pump housing (3) is disposed on the heat-preserving base (1) and is sealingly connected to the upper end edge of the heat-preserving base (1) through the second sealing connection mechanism, a gap is reserved between the heat-preserving base (1) and the pump housing (3) to form a second enclosed space, which is a water bath temperature control chamber; the culture medium overflowing from the top of the accommodation space forms a curtain-like fluid under the guidance of the second fluid channel, and the culture medium undergoes curtain-like oxygen exchange in the second fluid channel to increase the volumetric oxygen coefficient before contacting the upper wall of the water bath temperature control chamber to achieve temperature control of the culture medium.

15. The disposable fixed bed for adherent cell culture according to claim 14, characterized in that the top of the heat-preserving base (1) is provided with an outwardly flanging edge with multiple connecting through-holes, the side wall of the pump housing (3) is provided with a bent portion corresponding to the outwardly flanging edge, a plurality of screw sleeves are disposed on the bent portion, the bent portion of the pump housing (3) presses against the outwardly flanging edge of the heat-preserving base (1), and the inner surface of the bent portion is a bevel; the second sealing connection mechanism comprises: a second flexible sealing ring, a flexible washer (2) and a second bolt-nut assembly; the second flexible sealing ring is disposed between the bent portion of the pump housing (3) and the outwardly flanging edge of the heat-preserving base (1), the flexible washer (2) is disposed on the side of the outwardly flanging edge facing opposite to the bent portion, and the heat-preserving base (1), the flexible washer (2) and the pump housing (3) are fixed together by the second bolt-nut assembly.

16. The disposable fixed bed for adherent cell culture according to claim 14, characterized in that a plurality of recessed structures (1a) with equal heights are disposed on the bottom surface of the heat-preserving base (1) and symmetrically distributed with the central axis of the connecting column (3b) as the center.

17. The disposable fixed bed for adherent cell culture according to claim 1, characterized in that further comprising: a liquid inlet (17), a liquid outlet (18), a first electrode seat (6), a second electrode seat (7), and a third electrode seat (8);
the liquid inlet (17), the liquid outlet (18), the first electrode seat (6), the second electrode seat (7), and the third electrode seat (8) are respectively disposed on the upper cover plate (5).

18. The disposable fixed bed for adherent cell culture according to claim 17, characterized in that electrode through-holes are opened on the top wall of the second support seat (15) and the annular compression plate (9), the bottoms of the first electrode seat (6) and the third electrode seat (8) are respectively inserted into the corresponding electrode through holes of the annular compression plate (9) and are flush with the lower end surface of the annular compression plate (9), the bottom of the second electrode seat (7) passes through a limiting ring (19) and is inserted into the electrode through-hole of the second support seat (15), the upper end surface of the limiting ring (19) abuts against the stepped surface of the second electrode seat (7), and the lower end surface of the limiting ring (19) abuts against the top wall of the second support seat (15).

19. The disposable fixed bed for adherent cell culture according to claim 14, characterized in that the upper wall of the water bath temperature control chamber is disposed at an obtuse angle to the vertical direction to extend the action time of the water bath temperature control chamber on the culture medium while accelerating the reflux speed of the culture medium.
